(19) United States
(12) Patent Application Publication
(10) Pub. No.: US 2022/0323549 A1
(43) Pub. Date: Oct. 13, 2022

(54) OIL-BASED WOUND CARE COMPOSITIONS AND METHODS

(71) Applicant: GLOBAL HEALTH SOLUTIONS, INC., Westlake Village, CA (US)

(72) Inventor: Bradley BURNAM, Calabasas, CA (US)

(73) Assignee: GLOBAL HEALTH SOLUTIONS, INC., Westlake Village, CA (US)

(21) Appl. No.: 17/642,686

(22) PCT Filed: Sep. 12, 2020
(86) PCT No.: PCT/US2020/050596

§ 371 Date: Mar. 12, 2022

Related U.S. Application Data
(60) Provisional application No. 62/899,707, filed on Sep. 12, 2019.

(51) Int. Cl. A61K 38/39; A61K 31/14; A61K 31/155; A61K 47/44; A61K 9/00; A61P 17/02

(52) U.S. Cl. CPC A61K 38/39; A61K 31/14; A61K 31/155; A61K 47/44; A61K 9/0014; A61P 17/02

(57) ABSTRACT

Compositions and methods for wound care or the dressing or treatment of wounds in a subject in need thereof. The compositions include an oil-based carrier, a polar solvent comprising one or more polar antimicrobial agents, and collagen or a collagen-based material. In at least some instances, the polar solvent comprising the one or more polar antimicrobial agents and the collagen or collagen-based material are suspended in the oil-based carrier.

### CROSS-REFERENCE TO RELATED APPLICATIONS

This application is a U.S. National Phase Application of PCT/US2020/050596, filed Sep. 12, 2020, which claims the benefit of priority to U.S. Provisional Application Ser. No. 62/899,707, filed Sep. 12, 2019, entitled “Oil-Based Wound Care Compositions and Methods,” the entire contents of each of which are hereby incorporated by reference, for all purposes, in their entirety.

### FIELD

The present disclosure is broadly concerned with oil-based wound care compositions and methods for the treatment of wounds. The disclosure is also concerned with oil-based compositions for the treatment of wounds that include collagen and polihexanide biguanide (PHMB), as well as the use of such compositions as wound dressing or post-surgical dressings.

### BACKGROUND

Wound care in patients and animals is a common clinical challenge faced by the healthcare and veterinary industries. Wounds may include, for example, trauma wounds, burns, ulcers, lesions, abscesses, diabetic wounds, pressure sores or ulcers, and grafts or wounds resulting from surgical procedures and operations. Wounds may result from physical injury, surgical procedures and operations, heat or chemical burns, pressure on the skin, radiation, infections, immune system deficiencies, malnourishment, as well as various medical conditions such as vascular disorders and diabetes.

Collagen is known to improve wound healing and stimulate tissue growth and is well-tolerated at the wound site. In particular, collagen is thought to aid in the migration of fibroblasts and keratinocytes to the wound site thereby improving tissue growth in the wound bed. Improved collagen wound care compositions are desirable.

### DETAILED DESCRIPTION

The present disclosure provides compositions and methods for dressing and/or treating wounds in a subject. It has been unexpectedly discovered that the presently disclosed oil-based compositions comprising micronized or powdered collagen and one or more polar antimicrobial ingredients, such as polyhexamethylene biguanide (PHMB), suspended in an oil-based carrier, are especially effective in the treatment of wounds. In particular, it has been discovered that the presently disclosed compositions provide for improved penetration and absorption of collagen throughout the wound site, thereby enabling efficient delivery of collagen to the wound bed. The presently disclosed compositions may also enable delivery of collagen and PHMB through the skin graft layer and to the base of the graft thereby improving healing in skin graft or allograft patients. It has also been unexpectedly been found that presently disclosed PHMB and collagen compositions, when prepared according to the presently disclosed techniques, synergistically results in improved wound healing and lower incidence of infection as compared to separate administration of PHMB compositions and collagen compositions. Additionally, it has been found that the presently disclosed oil-based compositions are more effective in accelerating healing and preventing infection than solid substrate collagen-based wound dressings that contain PHMB or that are infused or impregnated with PHMB. In some instances, the oil-based carrier melts or otherwise liquefies once applied to the wound site due to the heat of the skin and wound, causing the release or increasing the availability of the PHMB and collagen to the wound site and aiding in the absorption and penetration of the collagen and PHMB by the wound site.

According to at least one aspect of the present disclosure, an oil-based composition for the treatment or dressing of a wound is provided. The composition may include an oil-based carrier, a polar solvent comprising one or more polar antimicrobial agents, and collagen or a collagen-based material. The polar solvent comprising one or more polar antimicrobial agents is suspended in the oil-based carrier. In at least some instances, the polar solvent comprising the one or more antimicrobial agents is dispersed in the oil-based carrier to form a stable suspension such that the solvent and polar antimicrobial agent does not separate from the oil-based carrier for at least six months. In at least some instances, the collagen or collagen-based material is suspended in the oil-based carrier and does not separate from the oil-based carrier for at least six months.

In at least some instances, the collagen or collagen-based material is in powdered form. In some cases, the collagen or collagen-based material is micronized collagen. The collagen or collagen-based material may be characterized by an average particle size of from about 5 microns to about 80 microns, or from about 20 microns to about 70 microns, or from about 5 microns to about 30 microns, or from about 10 microns to about 30 microns, or from about 15 microns to about 30 microns. In some instances, the collagen or collagen-based material is characterized by an average particle size of less than 20 microns or less than 30 microns.

The collagen or collagen-base material may be a material selected from extracellular matrix materials, micronized extracellular matrix, purified collagen, Type I collagen, Type II collagen, Type III collagen, Type X collagen, collagen fibers, collagen fibrils, micronized collagen, defibrillated collagen, coarse collagen bundles, non-crosslinked collagen, non-mineralized collage, collagen treated to control cross-linking (e.g., via chemical, thermal, photo, or radiation-induced cross-linking), collagen-glycosaminoglycan (GAG) mixtures, and any combination thereof

The collagen or collagen-based material may comprises from about 5 wt % to about 35 wt %, or from about 5 wt % to about 25 wt %, or from about 5 wt % to about 20 wt %, or from about 5 wt % to about 15 wt %, or from about 15 wt % to about 30 wt %, of the oil-based composition.

The oil-based based carrier may be selected from animal oil, vegetable oil, natural oil, synthetic oil, hydrocarbon oils, silicone oils, and semi-synthetic derivatives thereof, and any combination thereof. The oil-based carrier may also be petrolatum, cocoa butter, jojoba oil, olive oil, soybean oil, coconut oil, beeswax, lanolin wax, carnauba wax, stearic acid, or any mixture thereof. The oil-based carrier may also be mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C12-15 alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combination thereof.

In some instances, the polar solvent may be water, ethanol, or a mixture of ethanol and water. In some cases, the polar solvent may further include ascetic acid. In some aspects, the one or more polar antimicrobial agents comprises a cationic biocide. The cationic biocide may be benzalkonium chloride, cetrimide, chlorhexidine, polihexanide biguanide (polihexanide, polyhexamethylene biguanide, polyhexamethylene guanide, poly(iminoimidocarbonyl-iminoimidocarbonyl-iminohexamethylene), poly(hexamethylenebiguanide), polyaminopropyl biguanide), and salts or combinations thereof. The polar solvent may further include a preservative selected from the group consisting of benzalkonium chloride, cetrimide, chlorhexidine, and any combination thereof. In some instances, the composition comprises from about 0.001% to about 0.15% by weight benzalkonium chloride (BZK). In other instances, the composition comprises from about 0.001% to about 0.01% by weight or from about 0.005% to about 0.007% by weight benzalkonium chloride (BZK).

The presently disclosed compositions may further include one or more therapeutic agents selected from the group consisting of stem cells, TGF-alpha, TGF-beta (TGFβ1, TGFβ2, TGFβ3), platelet-derived growth factor (PDGF), epidermal growth factor (EGF), fibroblast growth factor also referred to as keratinocyte growth factor (FGF1, FGF2, FGF4, FGF7), vascular endothelial growth factor (VEGF), insulin-like growth factor (IGF), connective tissue growth factor (CTGF), activin, interleukin-1 (IL1α, IL1β, TNFα, GM-CSF, a powdered antibiotic, an antifungal agent, a hemostatic agent, cytokines, and hyaluronic acid.

According to at least one aspect of the present disclosure, the presently disclosed compositions may be prepared by a process that includes: a) dissolving the one or more polar antimicrobial agents in a polar solvent to give an antimicrobial agent solution; b) heating the oil-based carrier to a temperature sufficient to cause the oil-based carrier to melt or to a temperature sufficient to provide a oil-based carrier density capable of suspending a powdered collagen or a powdered collagen-based material, resulting in a melted oil-based carrier; c) mixing a powdered collagen or a powdered collagen-based material into the melted oil-based carrier to give a suspended collagen oil-based carrier composition; d) heating the antimicrobial agent solution to a temperature higher than the temperature of the melted oil-based carrier to give a heated antimicrobial solution; e) mixing the suspended collagen oil-based carrier composition and the heated antimicrobial solution to give a melted mixture; and f) cooling the melted mixture to give the oil-based composition. In some instances, the heated antimicrobial solution has a temperature that is about 1° C. to about 5° C. higher, or about 1° C. to about 10° C. higher, or about 1° C. to about 15° C. higher, than the temperature of the suspended collagen oil-based carrier composition at the time of mixing.

In some instances, the polar antimicrobial agent is polyhexamethylene biguanide (PHMB) and the oil-based carrier is petrolatum. In such instances, the PHMB may be dissolved in a polar solvent to form a PHMB solution and the PHMB solution dispersed in the petrolatum containing collagen. In such cases, the PHMB solution may be dispersed in the petrolatum containing collagen to form a stable suspension such that the PHMB solution does not separate from the petrolatum for at least six months. Additionally, the collagen or collagen-based material remains suspended in the petrolatum for at least six months. According to at least one aspect, the petrolatum-based collagen/PHMB compositions contain no emulsifier.

The presently disclosed petrolatum-based collagen/PHMB compositions may be prepared by a process that includes: a) dissolving the PHMB in a polar solvent to give a PHMB solution; b) heating the petrolatum to a temperature sufficient to cause the petrolatum to melt to give a melted petrolatum; c) adding collagen or a collagen-based material to the melted petrolatum to give a suspended collagen melted petrolatum mixture; d) heating the PHMB solution to a temperature higher than the temperature of the melted petrolatum to give a heated PHMB solution; e) mixing the suspended collagen melted petrolatum mixture and the heated PHMB solution to give a melted mixture; and f) cooling the melted mixture to give the petrolatum-based composition collagen/PHMB composition. In some instances, the PHMB solution is heated to a temperature that is about 1° C. to about 5° C. higher than the temperature of the suspended collagen melted petrolatum mixture. According to at least one aspect of the present disclosure, the resultant petrolatum-based collagen/PHMB composition does not require an emulsifier to form a stable suspension of collagen or PHMB dispersed in the petrolatum. Further, the petrolatum-based PHMB composition prepared according to this process does not require high shear mixing to form a stable suspension of collagen and PHMB in petrolatum in the absence of an added emulsifier.

In instances in which the oil-based carrier is petrolatum, the composition may include greater than about 60% by weight petrolatum, or greater than about 70% by weight petrolatum, or greater than about 80% by weight petrolatum, or greater than about 90% by weight petrolatum. According to one aspect, the presently disclosed compositions may include from about 0.1% to about 1% by weight PHMB, or from about 0.05% to about 5% by weight PHMB, or from about 0.05% to about 3% by weight PHMB, or from about 0.2% to about 0.6% by weight PHMB, or from about 0.3% to about 0.5% by weight PHMB, or from about 0.1% to about 3.5% by weight PHMB, or from about 0.05% to about 2.5% by weight PHMB, or from about 0.5% to about 3% by weight PHMB, or from about 0.5% to about 2.5% by weight PHMB, or from about 1.5% to about 2.5% by weight PHMB.

PHMB is closely related to the polymeric biguanide polyaminopropyl biguanide (PAPB). Therefore, in at least some instances, polyaminopropyl biguanide (PAPB) may be substituted for the PHMB in the presently disclosed compositions and methods. For example, the oil-based wound care compositions may include from about 0.005% to about 5% by weight PAPB, or from about 0.01% to about 5% by weight PAPB, or from about 0.05% to about 5% by weight PAPB, or from about 0.05% to about 3% by weight PAPB, or from about 0.1% to about 1% by weight PAPB, or from about 0.2% to about 0.6% by weight PAPB, or from about 0.3% to about 0.5% by weight PAPB, or from about 0.1% to about 3.5% by weight PAPB, or from about 0.05% to about 2.5% by weight PAPB, or from about 0.5% to about 3% by weight PAPB, or from about 0.5% to about 2.5% by weight PAPB, or from about 1.5% to about 2.5% by weight PAPB.

In general, the presently disclosed compositions are topical compositions suitable for application to the wound of a subject in need thereof In some instances, the presently disclosed compositions may take the form of an oil, or an ointment, or a cream suitable for topical administration to a wound site.

According to at least one aspect of the present disclosure, a method of treating or dressing a wound in a subject is provided. The method includes applying the presently disclosed compositions to a wound, post-surgical wound, or a post-surgical skin graft in need of dressing or treatment. The method may further include covering the composition with a wound covering selected from the group consisting of a bandage, wrap, gauze, sponge, and film, following the application of the composition to the wound or graft in need of treatment.

According to one aspect of the present disclosure, a method of treating or dressing a wound in a subject may include contacting the presently disclosed compositions to a wound covering, wherein the wound covering is a selected from the group consisting of a bandage, wrap, gauze, sponge, and film. The method may further include applying the wound covering to the wound or graft in need of treatment. Alternatively, the method may include impregnating the wound covering with the presently disclosed compositions and applying the wound covering to the wound or graft in need of treatment.

### EXAMPLES

The following examples are included to demonstrate preferred embodiments of the disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Formulation Example 1

An oil-based wound care composition is prepared in accordance with the present disclosure by dissolving a polar antimicrobial agent in a polar solvent to give a antimicrobial agent solution. Then an oil-based carrier is heated to a temperature sufficient to cause the oil-based carrier to melt or to a temperature sufficient to provide an oil-based carrier density capable of suspending a powdered collagen or a powdered collagen-based material, to produce a melted oil-based carrier. Next, a powdered collagen or a powdered collagen-based material is mixed into the melted oil-based carrier to give a suspended collagen oil-based carrier composition. The antimicrobial agent solution is then heated to a temperature higher than the temperature of the melted oil-based carrier to give a heated antimicrobial solution. Next, the suspended collagen oil-based carrier composition is mixed with the heated antimicrobial solution to give a melted mixture. Finally, the melted mixture is cooled to give the oil-based composition.

### Example 2

### Formulation Example 2

An oil-based wound care composition is prepared in accordance with the present disclosure by dissolving polyhexamethylene biguanide (PHMB) in water to give a PHMB antimicrobial agent solution. Then coconut oil is heated to 30° C., to produce a melted coconut oil-based carrier. Next, micronized collagen powder is mixed into the melted coconut oil-based carrier to give a suspended collagen coconut oil-based carrier composition. The average particle size of the micronized collagen is from about 5 microns to about 80 microns. The PHMB antimicrobial agent solution is then heated to 35° C. to give a heated PHMB antimicrobial solution. Next, the suspended collagen coconut oil-based carrier composition is mixed with the heated PHMB antimicrobial solution to give a melted mixture. Finally, the melted mixture is cooled to give the coconut oil-based composition that comprises from about 60 wt % to 90 wt % coconut oil, from about 5 wt % to about 35 wt % collagen, from about 0.05 wt % to about 5 wt % PHMB, and from about 1 wt % to about 15 wt % water.

### Example 3

### Formulation Example 3

An oil-based wound care composition is prepared in accordance with the present disclosure by dissolving polyhexamethylene biguanide (PHMB) and benzalkonium chloride (BZK) in water to give a PHMB/BZK antimicrobial agent solution. Then coconut oil is heated to 30° C., to produce a melted coconut oil-based carrier. Next, micronized collagen powder is mixed into the melted coconut oil-based carrier to give a suspended collagen coconut oil-based carrier composition. The average particle size of the micronized collagen is from about 5 microns to about 80 microns. The PHMB/BZK antimicrobial agent solution is then heated to 35° C. to give a heated PHMB/BZK antimicrobial solution. Next, the suspended collagen coconut oil-based carrier composition is mixed with the heated PHMB/BZK antimicrobial solution to give a melted mixture. Finally, the melted mixture is cooled to give the coconut oil-based composition that comprises from about 60 wt % to 90 wt % coconut oil, from about 5 wt % to about 35 wt % collagen, from about 0.05 wt % to about 5 wt % PHMB, from about 0.001 wt % to about 0.15 wt % BZK, and from about 1 wt % to about 15 wt % water.

### Example 4

### Formulation Example 4

An oil-based wound care composition is prepared in accordance with the present disclosure by dissolving chlorhexidine in water to give a chlorhexidine antimicrobial agent solution. Then coconut oil is heated to 30° C., to produce a melted coconut oil-based carrier. Next, micronized collagen powder is mixed into the melted coconut oil-based carrier to give a suspended collagen coconut oil-based carrier composition. The average particle size of the micronized collagen is from about 5 microns to about 80 microns. The chlorhexidine antimicrobial agent solution is then heated to 35° C. to give a heated chlorhexidine antimicrobial solution. Next, the suspended collagen coconut oil-based carrier composition is mixed with the heated chlorhexidine antimicrobial solution to give a melted mixture. Finally, the melted mixture is cooled to give the coconut oil-based composition that comprises from about 60 wt % to 90 wt % coconut oil, from about 5 wt % to about 35 wt % collagen, from about 0.05 wt % to about 5 wt % chlorhexidine, and from about 1 wt % to about 15 wt % water.

### Example 5

### Formulation Example 5

An oil-based wound care composition is prepared in accordance with the present disclosure by dissolving polyhexamethylene biguanide (PHMB) in ethanol to give a PHMB antimicrobial agent solution. Then coconut oil is heated to 30° C., to produce a melted coconut oil-based carrier. Next, micronized collagen powder is mixed into the melted coconut oil-based carrier to give a suspended collagen coconut oil-based carrier composition. The average particle size of the micronized collagen is from about 5 microns to about 80 microns. The PHMB antimicrobial agent solution is then heated to 35° C. to give a heated PHMB antimicrobial solution. Next, the suspended collagen coconut oil-based carrier composition is mixed with the heated PHMB antimicrobial solution to give a melted mixture. Finally, the melted mixture is cooled to give the coconut oil-based composition that comprises from about 60 wt % to 90 wt % coconut oil, from about 5 wt % to about 35 wt % collagen, from about 0.05 wt % to about 5 wt % PHMB, and from about 1 wt % to about 15 wt % ethanol.

### Example 6

### Formulation Example 6

An oil-based wound care composition is prepared in accordance with the present disclosure by dissolving polyhexamethylene biguanide (PHMB) in a mixture of 75% water and 25% ethanol to give a PHMB antimicrobial agent solution. Then coconut oil is heated to 30° C., to produce a melted coconut oil-based carrier. Next, micronized collagen powder is mixed into the melted coconut oil-based carrier to give a suspended collagen coconut oil-based carrier composition. The average particle size of the micronized collagen is from about 5 microns to about 80 microns. The PHMB antimicrobial agent solution is then heated to 35° C. to give a heated PHMB antimicrobial solution. Next, the suspended collagen coconut oil-based carrier composition is mixed with the heated PHMB antimicrobial solution to give a melted mixture. Finally, the melted mixture is cooled to give the coconut oil-based composition that comprises from about 60 wt % to 90 wt % coconut oil, from about 5 wt % to about 35 wt % collagen, from about 0.05 wt % to about 5 wt % PHMB, from about 0.75 wt % to about 11.25 wt % water, and from about 0.25 wt % to about 3.75 wt % ethanol.

### Example 7

### Formulation Example 7

An oil-based wound care composition is prepared in accordance with the present disclosure by dissolving polyhexamethylene biguanide (PHMB) in ascetic acid to give a PHMB antimicrobial agent solution. Then coconut oil is heated to 30° C., to produce a melted coconut oil-based carrier. Next, micronized collagen powder is mixed into the melted coconut oil-based carrier to give a suspended collagen coconut oil-based carrier composition. The average particle size of the micronized collagen is from about 5 microns to about 80 microns. The PHMB antimicrobial agent solution is then heated to 35° C. to give a heated PHMB antimicrobial solution. Next, the suspended collagen coconut oil-based carrier composition is mixed with the heated PHMB antimicrobial solution to give a melted mixture. Finally, the melted mixture is cooled to give the coconut oil-based composition that comprises from about 60 wt % to 90 wt % coconut oil, from about 5 wt % to about 35 wt % collagen, from about 0.05 wt % to about 5 wt % PHMB, and from about 1% to about 15% ascetic acid.

### Example 8

### Formulation Example 8

An oil-based wound care composition is prepared in accordance with the present disclosure by dissolving polyaminopropyl biguanide (PAPB) in water to give a PAPB antimicrobial agent solution. Then coconut oil is heated to 30° C., to produce a melted coconut oil-based carrier. Next, micronized collagen powder is mixed into the melted coconut oil-based carrier to give a suspended collagen coconut oil-based carrier composition. The average particle size of the micronized collagen is from about 5 microns to about 80 microns. The PAPB antimicrobial agent solution is then heated to 35° C. to give a heated PHMB antimicrobial solution. Next, the suspended collagen coconut oil-based carrier composition is mixed with the heated PAPB antimicrobial solution to give a melted mixture. Finally, the melted mixture is cooled to give the coconut oil-based composition that comprises from about 60 wt % to 90 wt % coconut oil, from about 5 wt % to about 35 wt % collagen, from about 0.05 wt % to about 5 wt % PAPB, and from about 1 wt % to about 15 wt % water.

### Example 9

### Formulation Example 9

An oil-based wound care composition is prepared in accordance with the present disclosure by dissolving polyhexamethylene biguanide (PHMB) in water to give a PHMB antimicrobial agent solution. Then carnauba wax is heated to 84° C., to produce a melted carnauba wax-based carrier. Next, micronized collagen powder is mixed into the melted carnauba wax-based carrier to give a suspended collagen carnauba wax-based carrier composition. The average particle size of the micronized collagen is from about 5 microns to about 80 microns. The PHMB antimicrobial agent solution is then heated to 87° C. to give a heated PHMB antimicrobial solution. Next, the suspended collagen carnauba wax-based carrier composition is mixed with the heated PHMB antimicrobial solution to give a melted mixture. Finally, the melted mixture is cooled to give the carnauba wax-based composition that comprises from about 60 wt % to 90 wt % carnauba wax, from about 5 wt % to about 35 wt % collagen, from about 0.05 wt % to about 5 wt % PHMB, and from about 1 wt % to about 15 wt % water.

### Example 10

### Formulation Example 10

An oil-based wound care composition is prepared in accordance with the present disclosure by dissolving polyhexamethylene biguanide (PHMB) in water to give a PHMB antimicrobial agent solution. Then beeswax is heated to 68° C., to produce a melted beeswax-based carrier. Next, micronized collagen powder is mixed into the melted beeswax-based carrier to give a suspended collagen beeswax-based carrier composition. The average particle size of the micronized collagen is from about 5 microns to about 80 microns. The PHMB antimicrobial agent solution is then heated to 72° C. to give a heated PHMB antimicrobial solution. Next, the suspended collagen beeswax-based carrier composition is mixed with the heated PHMB antimicrobial solution to give a melted mixture. Finally, the melted mixture is cooled to give the beeswax-based composition that comprises from about 60 wt % to 90 wt % beeswax, from about 5 wt % to about 35 wt % collagen, from about 0.05 wt % to about 5 wt % PHMB, and from about 1 wt % to about 15 wt% water.

### Example 11

### Formulation Example 11

An oil-based wound care composition is prepared in accordance with the present disclosure by dissolving polyhexamethylene biguanide (PHMB) in water to give a PHMB antimicrobial agent solution. Then petrolatum is heated to 42° C., to produce a melted petrolatum-based carrier. Next, micronized collagen powder is mixed into the melted petrolatum-based carrier to give a suspended collagen petrolatum oil-based carrier composition. The average particle size of the micronized collagen is from about 5 microns to about 80 microns. The PHMB antimicrobial agent solution is then heated to 45° C. to give a heated PHMB antimicrobial solution. Next, the suspended collagen petrolatum-based carrier composition is mixed with the heated PHMB antimicrobial solution to give a melted mixture. Finally, the melted mixture is cooled to give the petrolatum-based composition that comprises from about 60 wt % to 90 wt % petrolatum, from about 5 wt % to about 35 wt % collagen, from about 0.05 wt % to about 5 wt % PHMB, and from about 1 wt % to about 15 wt % water.

### Example 12

### Formulation Example 12

An oil-based wound care composition is prepared in accordance with the present disclosure by dissolving polyhexamethylene biguanide (PHMB) and benzalkonium chloride (BZK) in water to give a PHMB/BZK antimicrobial agent solution. Then petrolatum is heated to 42° C., to produce a melted petrolatum-based carrier. Next, micronized collagen powder is mixed into the melted petrolatum-based carrier to give a suspended collagen petrolatum-based carrier composition. The average particle size of the micronized collagen is from about 5 microns to about 80 microns. The PHMB/BZK antimicrobial agent solution is then heated to 45° C. to give a heated PHMB/BZK antimicrobial solution. Next, the suspended collagen petrolatum-based carrier composition is mixed with the heated PHMB/BZK antimicrobial solution to give a melted mixture. Finally, the melted mixture is cooled to give the petrolatum-based composition that comprises from about 60 wt % to 90 wt % petrolatum, from about 5 wt % to about 35 wt % collagen, from about 0.05 wt % to about 5 wt % PHMB, from about 0.001 wt % to about 0.15 wt % BZK, and from about 1 wt % to about 15 wt % water.

### Example 13

### Formulation Example 13

An oil-based wound care composition is prepared in accordance with the present disclosure by dissolving chlorhexidine in water to give a chlorhexidine antimicrobial agent solution. Then petrolatum is heated to 42° C., to produce a melted petrolatum-based carrier. Next, micronized collagen powder is mixed into the melted petrolatum-based carrier to give a suspended collagen petrolatum-based carrier composition. The average particle size of the micronized collagen is from about 5 microns to about 80 microns. The chlorhexidine antimicrobial agent solution is then heated to 45° C. to give a heated chlorhexidine antimicrobial solution. Next, the suspended collagen petrolatum oil-based carrier composition is mixed with the heated chlorhexidine antimicrobial solution to give a melted mixture. Finally, the melted mixture is cooled to give the petrolatum-based composition that comprises from about 60 wt % to 90 wt % petrolatum, from about 5 wt % to about 35 wt % collagen, from about 0.05 wt % to about 5 wt % chlorhexidine, and from about 1 wt % to about 15 wt % water.

### Example 14

### Formulation Example 14

An oil-based wound care composition is prepared in accordance with the present disclosure by dissolving polyhexamethylene biguanide (PHMB) in ethanol to give a PHMB antimicrobial agent solution. Then petrolatum is heated to 42° C., to produce a melted petrolatum-based carrier. Next, micronized collagen powder is mixed into the melted petrolatum-based carrier to give a suspended collagen petrolatum-based carrier composition. The average particle size of the micronized collagen is from about 5 microns to about 80 microns. The PHMB antimicrobial agent solution is then heated to 45° C. to give a heated PHMB antimicrobial solution. Next, the suspended collagen petrolatum-based carrier composition is mixed with the heated PHMB antimicrobial solution to give a melted mixture. Finally, the melted mixture is cooled to give the petrolatum-based composition that comprises from about 60 wt % to 90 wt % petrolatum, from about 5 wt % to about 35 wt % collagen, from about 0.05 wt % to about 5 wt % PHMB, and from about 1 wt % to about 15 wt % ethanol.

### Example 15

### Formulation Example 15

An oil-based wound care composition is prepared in accordance with the present disclosure by dissolving polyhexamethylene biguanide (PHMB) in a mixture of 75% water and 25% ethanol to give a PHMB antimicrobial agent solution. Then petrolatum is heated to 42° C., to produce a melted petrolatum-based carrier. Next, micronized collagen powder is mixed into the melted petrolatum-based carrier to give a suspended collagen petrolatum-based carrier composition. The average particle size of the micronized collagen is from about 5 microns to about 80 microns. The PHMB antimicrobial agent solution is then heated to 45° C. to give a heated PHMB antimicrobial solution. Next, the suspended collagen petrolatum-based carrier composition is mixed with the heated PHMB antimicrobial solution to give a melted mixture. Finally, the melted mixture is cooled to give the petrolatum-based composition that comprises from about 60 wt % to 90 wt % petrolatum, from about 5 wt % to about 35 wt % collagen, from about 0.05 wt % to about 5 wt % PHMB, from about 0.75 wt % to about 11.25 wt % water, and from about 0.25 wt % to about 3.75 wt % ethanol.

### Example 16

### Formulation Example 16

An oil-based wound care composition is prepared in accordance with the present disclosure by dissolving polyhexamethylene biguanide (PHMB) in ascetic acid to give a PHMB antimicrobial agent solution. Then petrolatum is heated to 42° C., to produce a melted petrolatum-based carrier. Next, micronized collagen powder is mixed into the melted petrolatum-based carrier to give a suspended collagen petrolatum-based carrier composition. The average particle size of the micronized collagen is from about 5 microns to about 80 microns. The PHMB antimicrobial agent solution is then heated to 45° C. to give a heated PHMB antimicrobial solution. Next, the suspended collagen petrolatum-based carrier composition is mixed with the heated PHMB antimicrobial solution to give a melted mixture. Finally, the melted mixture is cooled to give the petrolatum-based composition that comprises from about 60 wt % to 90 wt % petrolatum, from about 5 wt % to about 35 wt % collagen, from about 0.05 wt % to about 5 wt % PHMB, and from about 1% to about 15% ascetic acid.

### Example 17

### Formulation Example 17

An oil-based wound care composition is prepared in accordance with the present disclosure by dissolving polyaminopropyl biguanide (PAPB) in water to give a PAPB antimicrobial agent solution. Then petrolatum is heated to 42° C., to produce a melted petrolatum-based carrier. Next, micronized collagen powder is mixed into the melted petrolatum-based carrier to give a suspended collagen petrolatum-based carrier composition. The average particle size of the micronized collagen is from about 5 microns to about 80 microns. The PAPB antimicrobial agent solution is then heated to 45° C. to give a heated PHMB antimicrobial solution. Next, the suspended collagen petrolatum-based carrier composition is mixed with the heated PAPB antimicrobial solution to give a melted mixture. Finally, the melted mixture is cooled to give the petrolatum-based composition that comprises from about 60 wt % to 90 wt % petrolatum, from about 5 wt % to about 35 wt % collagen, from about 0.05 wt % to about 5 wt % PAPB, and from about 1 wt % to about 15 wt % water.

### Example 18

### Stability—3 Months.

Formulation Examples 1-17 will be packaged in tubes and subjected to an accelerated stability study. Portions of Formulation Examples 1-17 will be placed sideways in a 40° C.±2° C./75%±5% relative humidity (RH) storage chamber for different intervals to yield a period of three months. The product will be assessed for physical and analytical characteristics. Formulation Examples 1-17 will exhibit no separation of components for at least 3 months when stored at 40° C.±2° C. and 75%±5% relative humidity. Additionally, the collagen will remain suspended for at least 3 months when stored at 40° C.±2° C. and 75%±5% relative humidity. Further, the antimicrobial agent will remain effective for at least 3 months when stored at 40° C.±2° C. and 75%±5% relative humidity.

### Example 19

### Stability—6 Months.

Formulation Examples 1-17 will be packaged in tubes and subjected to an accelerated stability study. Portions of Formulation Examples 1-17 will be placed sideways in a 40° C.±2° C./75%±5% relative humidity (RH) storage chamber for different intervals to yield a period of six months. The product will be assessed for physical and analytical characteristics. Formulation Examples 1-17 will exhibit no separation of components for at least 6 months when stored at 40° C.±2° C. and 75%±5% relative humidity. Additionally, the collagen will remain suspended for at least 6 months when stored at 40° C.±2° C. and 75%±5% relative humidity. Further, the antimicrobial agent will remain effective for at least 6 months when stored at 40° C.±2° C. and 75%±5% relative humidity.

### Example 20

### Topical Administration of Formulation Examples 1-17 Will Improve Clinical Outcomes in Subjects Having a Wound

The effect of topical administration of the oil-based wound care compositions disclosed in Formulation Examples 1-17 will be studied using a randomized, double-blind clinical study. During the clinical study, a group of human subjects will be topically administered a composition from Formulation Examples 1-17. In particular, the oil-based wound care composition will be topically applied to a wound in need of dressing or treatment in a subject. Subjects receiving treatment using the compositions of Formulation Examples 1-17 are expected to have improved standard clinical outcomes. In particular, subjects receiving treatment according to the presently disclosed methods and techniques are expected to exhibit an improvement in wound healing rate and/or a reduction in incidence of infection at the wound site.

### Example 21

### Topical Administration of Formulation Examples 1-17 Will Improve Clinical Outcomes in Subjects Having a Post-Surgical Wound

The effect of topical administration of the oil-based wound care compositions disclosed in Formulation Examples 1-17 will be studied using a randomized, double-blind clinical study. During the clinical study, a group of human subjects will be topically administered a composition from Formulation Examples 1-17. In particular, the oil-based wound care composition will be topically applied to a post-surgical wound in need of dressing or treatment in a subject. Subjects receiving treatment using the compositions of Formulation Examples 1-17 are expected to have improved standard clinical outcomes. In particular, subjects receiving treatment according to the presently disclosed methods and techniques are expected to exhibit an improvement in wound healing rate and/or a reduction in incidence of infection at the wound site.

### Example 22

### Topical Administration of Formulation Examples 1-17 Will Improve Clinical Outcomes in Subjects Having a Skin Graft

The effect of topical administration of the oil-based wound care compositions disclosed in Formulation Examples 1-17 will be studied using a randomized, double-blind clinical study. During the clinical study, a group of human subjects will be topically administered a composition from Formulation Examples 1-17. In particular, the oil-based wound care composition will be topically applied to a post-surgical skin graft in need of dressing or treatment in a subject. Subjects receiving treatment using the compositions of Formulation Examples 1-17 are expected to have improved standard clinical outcomes. In particular, subjects receiving treatment according to the presently disclosed methods and techniques are expected to exhibit an improvement in wound healing rate and/or a reduction in incidence of infection at the skin graft site.

### Example 23

### Topical Administration of Formulation Examples 1-17 Will Improve Clinical Outcomes in Subjects Receiving a Wound Covering

The effect of topical administration of a wound covering comprising the oil-based wound care compositions disclosed in Formulation Examples 1-17 will be studied using a randomized, double-blind clinical study. During the clinical study, a group of human subjects will be topically administered a wound covering comprising the composition from Formulation Examples 1-17. In particular, the oil-based wound care compositions according to Formulation Examples 1-17 will be applied to a wound covering such as a bandage, wrap, gauze, sponge, or film. In some instances, the wound covering may be impregnated or saturated with the oil-based wound care compositions according to Formulation Examples 1-17. In other instances, the wound covering may be just contacted with the oil-based wound care compositions disclosed in Formulation Examples 1-17. The treated wound covering will be topically applied to a wound or post-surgical skin graft in need of dressing or treatment in a subject. Subjects receiving treatment using the wound coverings treated with the oil-based wound care compositions of Formulation Examples 1-17 are expected to have improved standard clinical outcomes. In particular, subjects receiving treatment according to the presently disclosed methods and techniques are expected to exhibit an improvement in wound healing rate and/or a reduction in incidence of infection at the wound or skin graft site.

What is claimed is:

1-62. (canceled)

63. An oil-based composition for the treatment or dressing of a wound, the composition comprising:
an oil-based carrier, the oil-based carrier comprising greater than about 60% by weight of the oil-based composition;
a polar solvent comprising one or more polar antimicrobial agents, wherein the polar solvent comprising one or more polar antimicrobial agents is suspended in the oil-based carrier; and
collagen or a collagen-based material, the collagen or collagen-based material comprising micronized collagen and comprising from about 5 wt % to about 35 wt % of the oil-based composition;
wherein the one or more polar antimicrobial agents comprises a cationic biocide selected from the group consisting of benzalkonium chloride, cetrimide, chlorhexidine, polihexanide biguanide (polihexanide, polyhexamethylene biguanide, polyhexamethylene guanide, poly(iminoimidocarbonyl-iminoimidocarbonyl-iminohexamethylene), poly(hexamethylenebiguanide), polyaminopropyl biguanide), and salts or combinations thereof.

64. The composition according to claim 63, wherein the collagen or collagen-based material is suspended in the oil-based carrier.

65. The composition according to claim 64, where in the collagen or collagen-based material is characterized by an average particle size of from about 5 microns to about 80 microns.

66. The composition according to claim 63, wherein the collagen or collagen-based material comprises a material selected from the group consisting of extracellular matrix materials, micronized extracellular matrix, purified collagen, Type I collagen, Type II collagen, Type III collagen, Type X collagen, collagen fibers, collagen fibrils, micronized collagen, defibrillated collagen, coarse collagen bundles, non-crosslinked collagen, non-mineralized collage, collagen treated to control cross-linking (e.g., via chemical, thermal, photo, or radiation-induced cross-linking), collagen-glycosaminoglycan (GAG) mixtures, and any combination thereof.

67. The composition according to claim 63, wherein the oil-based based carrier is selected from the group consisting of animal oil, vegetable oil, natural oil, synthetic oil, hydrocarbon oils, silicone oils, and semi-synthetic derivatives thereof, and any combination thereof.

68. The composition according to claim 63, wherein the oil-based based carrier is selected from the group consisting of is selected from the group consisting of mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C12-15 alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combination thereof

69. The composition according to claim 63, wherein the oil-based based carrier is coconut oil.

70. The composition according to claim 64, wherein the oil-based based carrier is petrolatum.

71. The composition according to claim 63, wherein the polar solvent is selected from the group consisting of water, ethanol, and any combination thereof

72. The composition according to any to claim 63, wherein the one or more polar antimicrobial agents comprises polyhexamethylene biguanide (PHMB).

73. The composition according to claim 72, wherein the composition comprises from about 0.05% to about 5% by weight PHMB.

74. The composition according to claim 63, wherein the polar solvent further comprises a preservative selected from the group consisting of benzalkonium chloride, cetrimide, chlorhexidine, and any combination thereof.

75. The composition according to claim 74, wherein the composition comprises from about 0.001% to about 0.15% by weight benzalkonium chloride (BZK).

76. The composition according to any one of claims 1-48, wherein the composition excludes an added emulsifier.

77. A method of treating or dressing a wound in a subject, the method comprising applying the composition according to claim 63 to a wound in need of dressing or treatment.

78. A method of treating or dressing a skin graft post-surgery in a subject in need thereof, the method comprising applying the composition according to claim 63 to a post-surgical skin graft in need of dressing or treatment.
